(19) United States
(12) Patent Application Publication
(10) Pub. No.: US 2015/0136701 A1
(43) Pub. Date: May 21, 2015

(54) METHODS AND DEVICES FOR TREATING A DISEASE STATE

(71) Applicant: ARNON CHAIT, Bay Village, OH (US)

(72) Inventor: ARNON CHAIT, Bay Village, OH (US)

(21) Appl. No.: 14/082,649

(22) Filed: Nov. 18, 2013

(51) Int. Cl. A61M 1/36; A61M 1/34; B01D 15/38

(52) U.S. Cl. CPC A61M 1/3687; B01D 15/3809; A61M 1/3612; A61M 1/34; A61M 1/361; A61M 2202/0464; A61M 2205/04; A61M 2205/3327; A61M 2205/75
USPC 210/662; 623/11.11; 210/660; 210/663

(57) ABSTRACT

The invention discloses devices and methods for allowing selective removal of a protein solution from a human biological fluid. In some embodiments, a column with specificity of binding for a predetermined protein is employed to remove said protein to prevent onset of a disease or for prevention for the continued progress of the same disease. The invention has particular application in neurodegenerative disorders.

The present application seeks priority from U.S. Patent Application 61/729,503 of similar inventorship, filed on 25 Nov. 2012.

### FIELD AND BACKGROUND OF THE INVENTION

The present invention, in some embodiments thereof, relates to methods and devices for selectively removing predetermined proteins associated with protein aggregation-related illnesses. The instant invention, in some embodiments, allows for a selected biological fluid to be filtered through a protein-specific column to reduce the concentration of the protein prone to aggregation.

One of the greatest challenges facing many US families is a parent or grandparent with a neurodegenerative disorder. It is estimated that 15 million families expend in excess of $1 billion each year to care for family members who have been afflicted by debilitating diseases like Alzheimer's. While the causes of such diseases are being better defined and while drugs are in various stages of testing and development, millions suffer the effects of dementia and/or loss of motor control.

In recent years, it has been noted that neurodegenerative and other diseases often show a certain behavior, namely a certain protein self-aggregates to form plaques, fibers or other structures which may be some of the causative features of the relevant disease states. The proteins are generally not mutated and their structures may be in the native form or be modified/mis-folded; under physiological conditions of heightened protein concentration and/or folding errors, the proteins leave solution for a thermodynamically more stable aggregated arrangement which may be causative of various disease states. It should be noted that the dynamic process of protein aggregation may involve a plethora of interacting aggregates of different size, forming a dynamic size distribution function, with potentially only some of its members involved in the observed toxicity and disease pathology.

Effective treatments for Parkinson's, Alzheimer's and other debilitating diseases are often lacking or may aid in ameliorating some aspects of the illness but not providing a cure. The challenge of “healthy” proteins leaving their respective solutions to form unwanted superstructures is not trivial, as the protein monomers are potentially needed for proper biological function. One needs to treat a disease without harming vital proteins. While the exact biological origins and cause of such diseases are presently unknown and are under active study, their collective presentation of certain proteins in an aggregated state could indeed be related to the pathological course of aggregation-related diseases. More recently, customized biological therapies that include antibodies targeting such aggregation-prone proteins to reduce their ability to aggregate in vivo have been proposed, further pointing to the potential utility of other methods and techniques to accomplish the same.

European patent EP0921790A1 to Moss provides a pharmaceutical composition, comprising a sulfonyl fluoride and a pharmaceutically acceptable carrier. Also provided is a method of treating Alzheimer's disease in an individual in need of such treatment, comprising the step of administering to said individual a therapeutically effective dose of methanesulfonyl fluoride. Further provided is a method of enhancing cognitive performance in an individual in need of such treatment, comprising the step of administering to said individual a therapeutically effective dose of methanesulfonyl fluoride.

European patent EP1325338A2 provides methods and compositions for screening, diagnosis and prognosis of Alzheimer's disease, for monitoring the effectiveness of Alzheimer's disease treatment, and for drug development. Alzheimer's Disease-Associated Features (AFs), detectable by two-dimensional electrophoresis of cerebrospinal fluid, serum or plasma are described. The invention further provides Alzheimer's Disease-Associated Protein Isoforms (APIs) detectable in cerebrospinal fluid, serum or plasma, preparations comprising isolated APIs, antibodies immunospecific for APIs, pharmaceutical compositions, diagnostic and therapeutic methods, and kits comprising or based on the same.

U.S. Pat. No. 7,189,703 to Balin, et al. teaches a method of treating Alzheimer's disease in a mammal comprising administering to the mammal an anti-microbial agent having anti-Chlamydia pneumoniae activity. The invention also relates to a method of diagnosing Alzheimer's disease in a mammal comprising measuring the serum anti-Chlamydia pneumoniae antibody titer in a patient suspected of having Alzheimer's disease.

European patent EP0575048A1 teaches a method for treating patients having neurodegenerative diseases, and in particular, to a method for treating patients suffering from those diseases which cause a cholinergic deficit.

European patent EP2145628A1 describes small interfering RNAs, short hairpin RNAs and vectors and pharmaceutical compositions comprising the siRNA and shRNAs. Methods for treating a neurodegenerative disorder, namely SCA-1 are also described. Particularly, the invention provides a small interfering RNA consisting of a first and second strand wherein said first strand is encoded by a DNA sequence comprising SEQ ID NO. 1 or SEQ ID NO. 2.

U.S. Pat. No. 7,105,183 to McGrath teaches methods of treating a macrophage-associated neurodegenerative disease such as amyotrophic lateral sclerosis (ALS), Alzheimer's disease (AD), or multiple sclerosis (MS) in a subject by administering chlorite in an amount effective to decrease blood immune cell activation. The invention also features methods of monitoring therapy by assessing blood immune cell activation before and after therapy.

The prior art generally describes methods for treating an existing condition through the application of chemicals and biological reagents.

### SUMMARY OF THE INVENTION

It is therefore a purpose of the present invention, in some embodiments, to describe methods and devices externally reducing the concentration of a protein to prevent the onset or the spread of a predetermined medical condition.

The invention includes a method for treating a disease in a patient, including the following: determining that a patient has an in vivo concentration of at least one predetermined protein above a predetermined threshold concentration, wherein the protein may be associated with disease; creating at least two separated ports on the patients, allowing access to at least one biological fluid; allowing a predetermined biological fluid from the patient to be directed from a first of the ports towards a sterile column, the column packed with a stationary phase capable of binding the predetermined protein at a predetermined level of specificity; letting the fluid pass through the column for a predetermined period of time, thereby causing the concentration of the at least one predetermined protein in the fluid to be lowered below a predetermined threshold; allowing the fluid to return to the patient through a second of the ports after fluid passage through the column; and, closing the ports after the predetermined period of time has elapsed and the fluid has been returned to the patient.

In one aspect of the method, the disease is Alzheimer's disease.

In another aspect of the method, the disease is Parkinson disease.

In another aspect of the method disease is Huntington disease

In another aspect of the method, the disease is Amyotropic Lateral Sclerosis disease

In another aspect of the method, the disease is a neurodegenerative disease.

In another aspect of the method, the disease is selected from the list: Alzheimer's disease, aortic medial amyloid, atherosclerosis, cardiac arrhythmias, isolated atrial amyloidosis, cerebral amyloid angiopathy, diabetes mellitus type 2, dialsysis-related amyloidosis, familial amyloid polyneuropathy, Finnish amyloidosis, hereditary non-neuropathic systemtic amyloidosis, Huntington's disease, lattice corneal dystrophy, medullary carcinoma of the thyroid, multiple myeoloma, Parkinson's disease, prolactinomas, rheumatoid arthritis, sporadic inclusion body myositis, systemic AL amyloidosis, transmissible spongiform encephalopathy.

In another aspect of the method, the predetermined protein is beta-amyloid.

In another aspect of the method, the predetermined protein is tau.

In another aspect of the method, the predetermined protein is alpha-synuclein.

In another aspect of the method, the stationary phase includes immobilized antibodies specific for interaction and binding of the predetermined protein.

In another aspect of the method, the step of determining is performed in advance of the step of creating the access ports.

In another aspect of the method, the threshold is below a concentration value required for protein aggregation.

In another aspect of the method, the predetermined period of time is 3, 6, 12, or 24 hours.

In another aspect of the method, the step of measuring the concentration of the predetermined protein before the step of closing the ports.

In another aspect of the method, the predetermined protein is realized as a plurality of unique proteins.

In another aspect of the method, a pump is employed in the step of letting the biological fluid pass through the column and a sterile filter is placed after the column.

The invention additionally includes a device for treatment of aggregation-related disease, including: a diagnostic device for determining the in vivo concentration of a predetermined protein in a predetermined biological fluid in a patient, wherein the protein may be associated with onset of the aggregation-related disease; a column, wherein the stationary phase of the column is adapted to interact with and bind the predetermined protein at a predetermined level of specificity; and, tubing, wherein the tubing is adapted to allow for the biological fluid from a port on the patient to flow through the column and from the column back to at least a second port at a different location on the patient.

In one aspect of the device, the disease is Alzheimer's disease.

In another aspect of the device, the predetermined protein is beta amyloid.

In another aspect of the device, the predetermined protein is tau.

In another aspect of the device, the stationary phase is adapted in part to include immobilized antibodies or other binding elements with predetermined specificity of binding for the predetermined protein.

In another aspect of the device, the predetermined protein is realized as a plurality of unique proteins.

The invention also includes a method for prophylactic treatment for Alzheimer's disease, including the following: determining that a patient has an in vivo cerebrospinal concentration of at least one predetermined protein above a predetermined threshold, wherein the protein is associated with Alzheimer's onset and the threshold is beneath the self-aggregation concentration for the protein; anaesthetizing the patient; making at least two incisions in the region of the brain of the patient; allowing cerebrospinal fluids from the brain of the patient to be directed from a first incision towards a sterile column, the column packed with a stationary phase capable of binding the predetermined protein at a predetermined level of specificity; letting the cerebrospinal fluid pass through the column for a predetermined period of time, wherein the fluid is sterile filtered after passage through the column and prior to return of the fluid to the brain of the patient through a second incision; and, closing the incisions after the predetermined period of time has elapsed and the fluid has been returned to the patient.

In one aspect of the method, the determining is performed on a patient age of 40 or higher.

In another aspect of the method, the method is performed on a patient more than once in his life.

In another aspect of the method, the method is performed every five or ten years.

The invention provides for a method for treating a disease in a patient, including the following: determining that a patient has an in vivo concentration of at least one predetermined protein above a predetermined threshold concentration, wherein the protein may be associated with disease; implanting a chamber in the body of the patient, wherein the chamber allows for flow of a bodily fluid through the chamber; allowing a predetermined biological fluid from the patient to be directed to flow through the chamber, the chamber including a stationary phase capable of binding the predetermined protein at a predetermined level of specificity; letting the fluid pass through the chamber for a predetermined period of time, thereby causing the concentration of the at least one predetermined protein in the fluid to be lowered below a predetermined threshold; and, removing the chamber.

In one aspect of the method, there is additionally at least one in vivo pump to direct the flow of the bodily fluid through the chamber.

In another aspect of the method, there is additionally at least one in vivo sensor to monitor the concentration of the at least one predetermined protein.

Unless otherwise defined, all technical and/or scientific terms used herein have the same meaning as commonly understood by one of ordinary skill in the art to which the invention pertains. “Protein”, “column”, “stationary phase”, “liquid phase”, “incision”, “port”, “antibody”, the specific diseases and specific proteins mentioned, and all other terms not defined otherwise may have their meaning as generally understood in the relevant arts.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. It is noted that similar elements in various drawings will have the same number, advanced by the appropriate multiple of 100.

In the drawings:

FIG. 1 shows a schematic view of an embodiment of the instant invention;

FIG. 2 shows an additional view of the first embodiment of the invention;

FIG. 3 shows a schematic view of an additional embodiment of the invention;

FIG. 4 shows a method associated with an embodiment of the invention;

FIG. 5 shows an additional method associated with an embodiment of the instant invention; and,

FIG. 6 shows a schematic view of an in vivo embodiment of the instant invention.

### DESCRIPTION OF SPECIFIC EMBODIMENTS OF THE INVENTION

The present invention, in some embodiments thereof, relates to a cover for touch-sensitive screens, wherein the cover provides both strong protective features for the fragile screen, while concomitantly providing full access to the screen and its touch-sensitive functionality.

For purposes of better understanding, some embodiments of the present invention are illustrated in the figures of the drawings. Without being bound by any theory, the following discussion is offered.

### First Embodiment

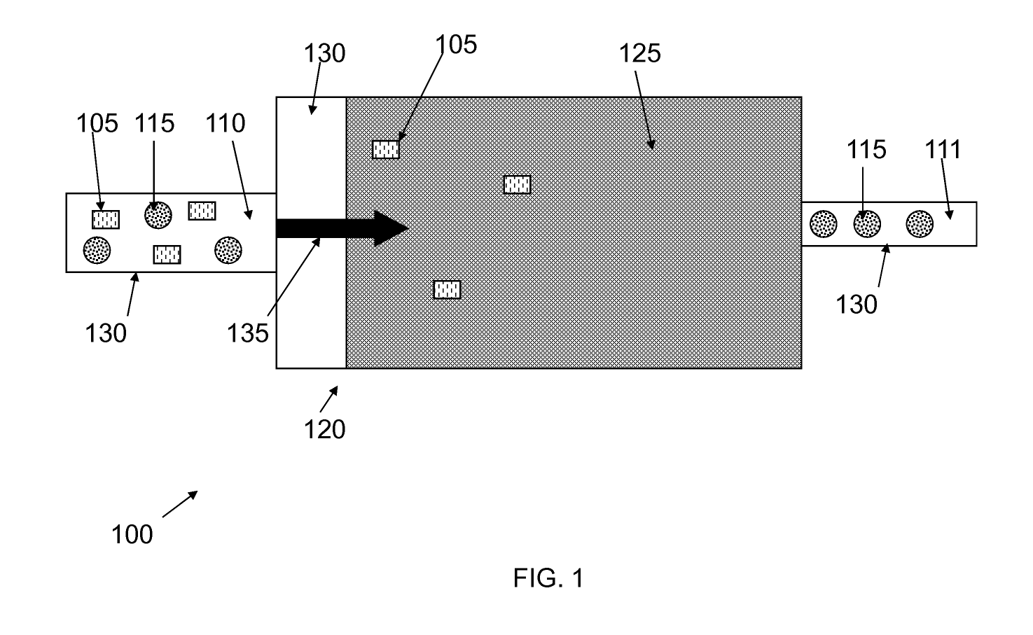

Attention is turned to FIG. 1 which shows a schematic view of an embodiment of an embodiment of the instant invention. Many disease states would appear to have associated with them proteins whose modified in vivo behavior may either be causative or associative with respect to the disease. Amyloids are fibrous structures resulting from the interaction of improperly folded proteins and peptides. Table I a partial list of diseases and associated proteins as recorded at http://en.wikipedia.org/wiki/Amyloid.

TABLE I

| | | Official |
|---|---|---|
| Disease | Protein featured | abbreviation |
| Alzheimer's disease | Beta amyloid^{[7][8][9]} | Aβ |
| Diabetes mellitus type 2 | IAPP (Amylin)^{[10][11]} | AIAPP |
| Parkinson's disease | Alpha-synuclein^{[8]} | none |
| Transmissible spongiform | PrP^{Sc[12]} | APrP |
| encephalopathy e.g. Bovine | | |
| spongiform encephalopathy | | |
| Huntington's Disease | Huntingtin^{[13][14]} | none |
| Medullary carcinoma of the thyroid | Calcitonin^{[15]} | ACal |
| Cardiac arrhythmias, Isolated atrial | Atrial natriuretic factor | AANF |
| amyloidosis | | |
| Atherosclerosis | Apolipoprotein AI | AApoA1 |
| Rheumatoid arthritis | Serum amyloid A | AA |
| Aortic medial amyloid | Medin | AMed |
| Prolactinomas | Prolactin | APro |
| Familial amyloid polyneuropathy | Transthyretin | ATTR |
| Hereditary non-neuropathic | Lysozyme | ALys |
| systemic amyloidosis | | |
| Dialysis related amyloidosis | Beta 2 microglobulin | Aβ2M |
| Finnish amyloidosis | Gelsolin | AGel |
| Lattice corneal dystrophy | Keratoepithelin | AKer |
| Cerebral amyloid angiopathy | Beta amyloid^{[16]} | Aβ |
| Cerebral amyloid angiopathy | Cystatin | ACys |
| (Icelandic type) | | |
| systemic AL amyloidosis | Immunoglobulin light | AL |
| | chain AL^{[15]} | |
| Multiple Myeloma | | |
| Sporadic Inclusion Body Myositis | S-IBM | none |

Self-aggregation of proteins is a type of phase transition—from a monomeric, soluble state into a multimeric stage. In the present invention, self-aggregation is further differentiated from specific protein-protein interactions, which characterize many higher-order structures of functional proteins (so-called 4D structure vs. the 3D structure of the monomeric form of the protein). Formation of functional 4D protein structures typically involve strong fixed covalent bonding and furthermore is commonly limited to a small number of monomeric units, e.g., up to 4 or 6 units arranged in a highly specific spatial arrangement. Moreover, formation of functional protein structures does not depend on the concentration of the monomeric protein, and sometimes involves other proteins, etc.

The dynamics of formation of non-specific self-aggregation of protein could be understood by classifying intermolecular interactions into two primary types: in a weakly ionized colloidal particle solution (biological fluid), long-range electrostatic interactions are combined with short range complex mix of interactions such as hydrophobic interactions, and the like. The entire dynamics of transitioning from a colloidal solution of monomeric proteins to one characterizes by aggregates growing in size in time, could be described by the interplay between these two broad types of intermolecular forces. This dynamic is also influenced by random fluctuations due to thermally-induced Brownian motions. As the monomeric concentration increases at a given degree of random kinetic energy level (corresponding to the solution temperature), the probability of self-interaction and aggregation increases.

For any degree of monomeric protein concentration there is a corresponding critical nucleus size of the aggregate form. The concept of a critical size is quite simple to describe: above that size, the nucleus will grow, and below that size, it will break down back into its monomeric sub-units. At low level of monomeric concentration the critical size is very large and it is improbable that the random motions of individual proteins would be sufficient to bring together enough proteins to form the required large critical aggregate size. But as solution concentration increases, the critical radius decreases, and the probability of observing a collection of monomeric proteins in a form of a nucleus with size greater than the critical threshold also increases. Although the process described varies continuously over the monomeric concentration range, there is typically a rather sharp transition from monomeric solution to a growing distribution of aggregates when a certain critical concentration level is reached at the saturation point. Above the saturation point, the probability of observing growing nuclei is greater than unity, and the growth dynamics of the entire population of aggregates is subsequently governed by a highly complex and self-interacting process.

Detailed observations and clinical studies have clearly correlated disease-specific proteins in their aggregate forms with the onset and progression of many neurodegenerative and other diseases. A classic example is the prion protein, which exists in two 3D structural forms: soluble conformation PrPc, and non-soluble conformation called PrPsc. The latter has been shown to be able to convert the former into the non-soluble form directly, thereby increasing its concentration until saturation is reached and aggregates are formed. The time period between presumed infectivity and saturation could be very long, sometimes decades, but once the saturation point is reached, the neuropathology of the disease is quite rapid, reflecting the underlying growth dynamics of aggregates in accordance with well-established laws of physics, as described by the Fokker-Plank and diffusion equations, and the Zeldovich nucleation rate. At the present time the neurological damage is not well understood in terms of a specific aggregate size, although the clinical failure of many Alzheimer's drugs that target late-stage large aggregates implicitly acknowledges the toxic role of intermediate smaller aggregates of, e.g., beta-amyloid or tau proteins. Regardless of which specific part of the dynamic aggregate size distribution function is related to the biological degenerative process itself, reducing the concentration of the disease-associated protein in the relevant biological fluid below that which corresponds to its in-vivo saturation limit should at least result in subsequent collapse of aggregate formation.

Selective filtration of specific proteins in a mixture could be efficiently accomplished by multiple techniques, which in the present invention will be collectively and generally referred to as column chromatography. Chromatography is a technique used in analytical chemistry, preparative production of DNA recombinant proteins, and related fields. Essentially, passing a mixture of soluble constituents through a column packed with a stationary phase (e.g., small silica particles for example) may afford certain degree of separation of the mixture components based on differential interaction of solution components with the preselected stationary phase. For example, smaller particles in the mixture may spend more time in the small spaces between the particles of the stationary phase while larger particles will typically exit the column much faster. This allows separation of mixture components based on their size. Other properties of mixture components may be exploited in a chromatography process for separation, including charge, affinity to other moieties placed on the stationary phase, hydrophobicity, and other properties.

One means to deploy a chromatography process to rid a sample of a specific protein from a biological fluid is to bind protein-specific moieties to the stationary phase. Stationary phase comprised of packed particles decorated with protein A is commonly used to purify the highly complex proteome resulting from the fermentation process of bacteria or cells in the production of recombinant antibodies, since protein A binds antibodies. Antibodies that specifically recognize beta-amyloid or alpha-synuclein could be bound to the stationary phase to selectively remove said proteins from a biological fluid that is passed through the column under conditions which encourage and support interaction between the antibodies and their target protein antigen. In this manner, the concentration of any specific protein could be reduced by passing it through a chromatography process that utilizes a stationary phase with attached binding elements such as antibodies that specifically recognize said protein.

Another related but different technique to remove waste products and excess water from blood is hemodialysis or peritoneal dialysis. Dialysis works by exploiting diffusion through concentration gradients that are formed by a semi-permeable membrane, in essence a type of efficient size-exclusion chromatography column. This process mimics the action of the kidneys in the body. Of course, there is no parallel in-vivo process for removal of specific disease-associated proteins. One embodiment of the present invention as described below represents an ex-vivo process for achieving this goal and relies on specific binding and removal of problematic protein species and not mere diffusion through concentration gradients.

FIG. 1 shows a system 100 for selectively removing a predetermined protein 105 from a biological fluid 110, while not affecting non-target proteins 115. The system includes a column 120 that includes a stationary phase 125 and a liquid phase 130 (shown separately for ease of understanding). The stationary phase 125 is adapted to selectively bind target protein 105 and let non-target proteins 115 to pass through the column 120. Additionally, there is tubing 130 before and after the column 120 which is run in the direction as shown 135. Target protein 105 is retained on the column 120 to allow for passage of post-column biological fluid 111 including non-target proteins 115 and other fluid components like cells, fats, and sugars. Post-column biological fluid 111 with non-target proteins 115 may be returned to a patient or may be run through the column 120 additional times so as to reduce the concentration of target protein 105 as much as possible.

An example of employment of this embodiment could be for the removal of beta-amyloid or other proteins from cerebrospinal fluid. The target protein 105 or proteins would be retained by the stationary phase 125 of the column 120 while cerebrospinal fluid 110 would pass through the column 120 with all non-target proteins 115. The tubing 130 would be adapted to bring cerebrospinal fluid 110 to the column and to return post-column cerebrospinal fluid 111 to the patient (not shown). Diagnostic tests could be run at any time to determine the concentration of target protein before, during and after treatment of cerebrospinal fluid 110 & 111 with the column 120 as shown in FIG. 1.

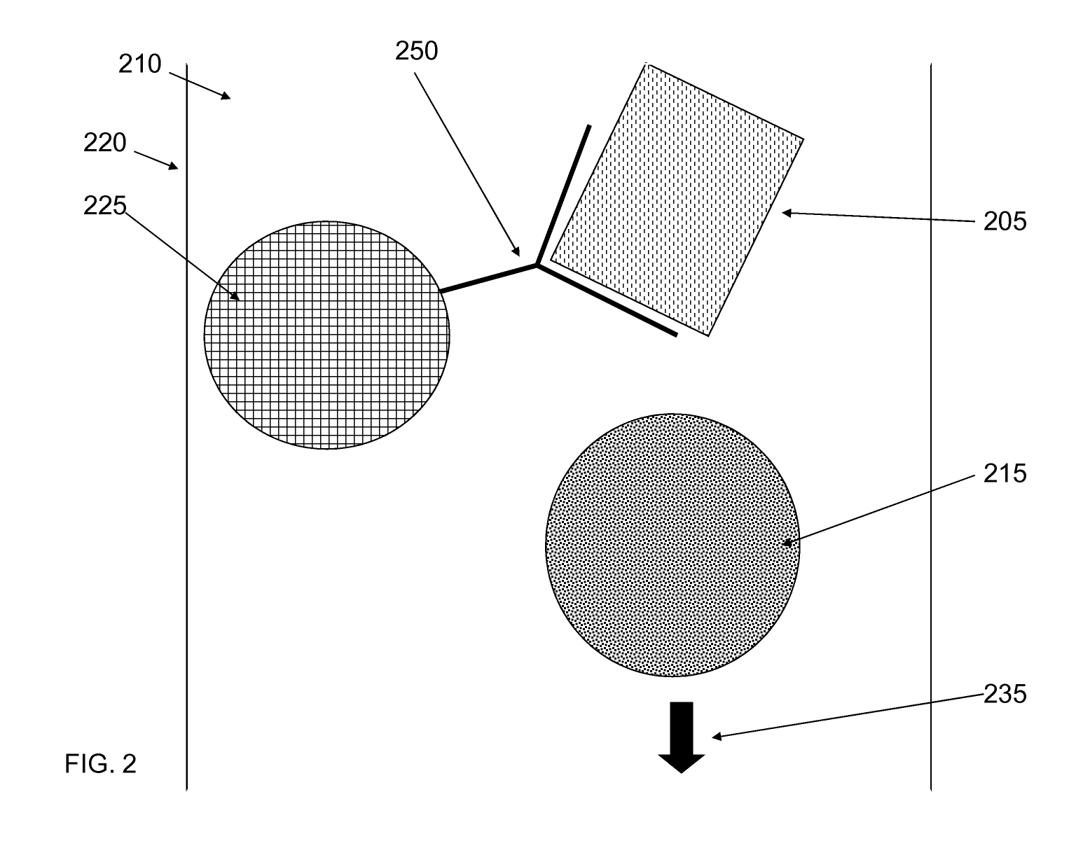

Attention is turned to FIG. 2 which shows a schematic view of a feature of the instant invention. The column 220 includes a stationary phase 225 adapted and modified to include immobilized binding agents 250 that may selectively bind target protein 205. Stationary phase 225 may be fully or partially modified to include binding agents 250 on the resin material used for column 220 packing. Binding agents 250 may be realized as antibodies, receptors or other molecules that show some level of specificity of interaction with predetermined target protein 205, either in its native form or in a modified form related to a disease state. Non-target protein 215 is not retained by the column 220 and freely continues 235 out of the stationary phase 225. The stationary phase 225 may be composed of any relevant column material such as Sepharose® or Sephadex® which are easily modified to bind active antibodies. The stationary phase 225 may have electrostatic and/or hydrophobic properties to further allow for specific binding and retention of target protein 205 and release of non-target 215 species. The target protein 205 may be a plurality of proteins, in which case multivalent antibodies or multiple distinct antibodies may be employed in the role of binding agents 250 for the target proteins. The liquid phase of the column 220 is selected to be sterile and biologically compatible to the fluid 210 being selectively cleaned. The column 220 is packed, maintained and run under conditions so as not to interfere with target protein 205 binding and facile non-target protein 215 passage 235 from the column 220.

### Second Embodiment

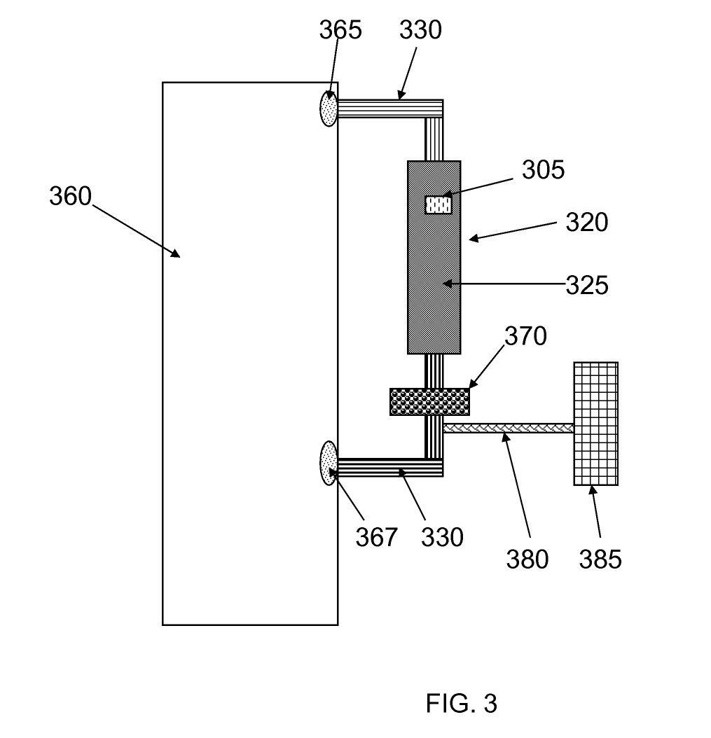

Attention is now turned to FIG. 3 which shows a schematic view of an embodiment of the instant invention. A region 360 of a patient is selected for treatment. A first port 365 is created generally with an incision followed by closure with a sterile stopcock that may be opened and closed at will. Tubing 330 is attached to the port 365 and is directed towards a column 320 which includes a stationary phase 325 modified to allow for binding of a predetermined protein 305. Fluid from the first port 365 travels through the tubing 330 and into the column 320, where protein 305 is retained; remaining components of the fluid passes through the column 320 and out to tubing 330 where it passes through a filter 370. A shunt 380 may optionally take a portion of the fluid to a diagnostic device 385 to determine the concentration of the target protein 305 after passage of the fluid through the column 320. Remaining fluid is returned through a second port 367 to the region 360 of the patient from which it was originally taken. Fluid travel time out of the body may be measured in minutes to hours and an optional pump (not shown) may be employed to control fluid flow rates through the column 320. If the diagnostic device 385 suggests that target protein 305 is at a concentration still higher than desired, then additional passes of fluid through the column 320 by way of the ports 365 & 367 may be performed.

### Third Embodiment

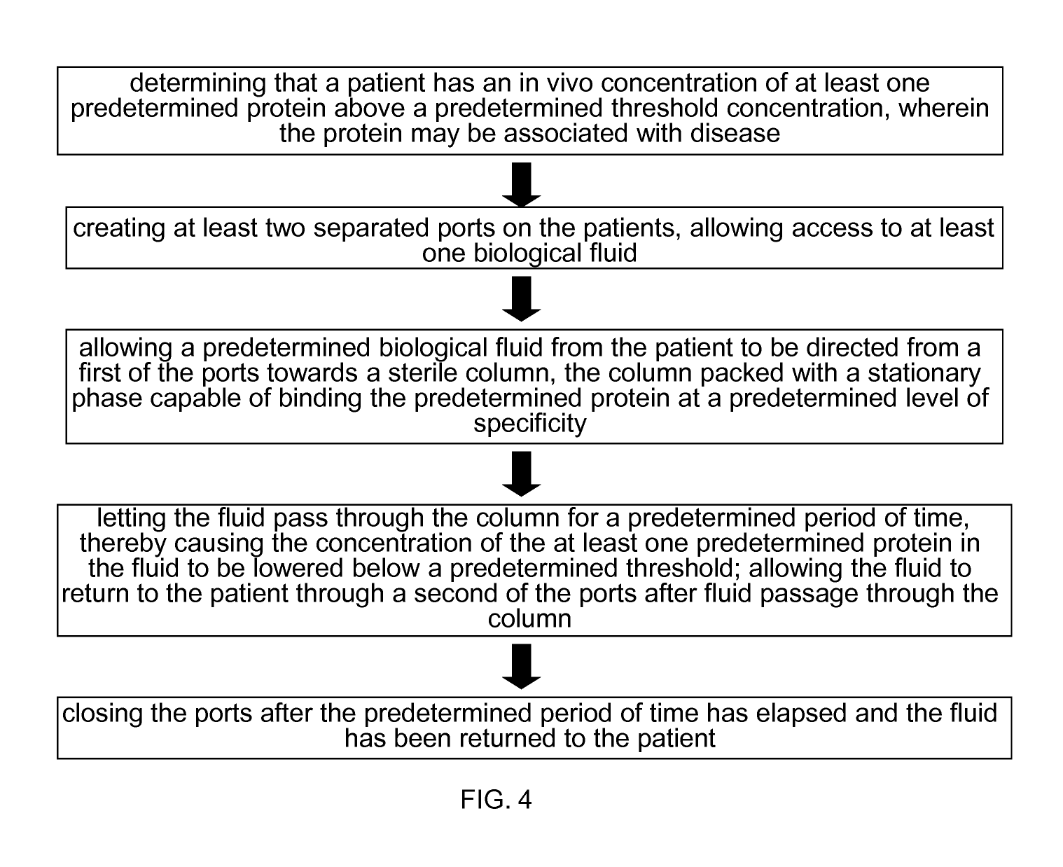

Attention is now turned to FIG. 4 which describes a method associated with an embodiment of the invention. The method includes the following: determining that a patient has an in vivo concentration of at least one predetermined protein above a predetermined threshold concentration, wherein the protein may be associated with disease; creating at least two separated ports on the patients, allowing access to at least one biological fluid; allowing a predetermined biological fluid from the patient to be directed from a first of the ports towards a sterile column, the column packed with a stationary phase capable of binding the predetermined protein at a predetermined level of specificity; letting the fluid pass through the column for a predetermined period of time, thereby causing the concentration of the at least one predetermined protein in the fluid to be lowered below a predetermined threshold; allowing the fluid to return to the patient through a second of the ports after fluid passage through the column; and; closing the ports after the predetermined period of time has elapsed and the fluid has been returned to the patient. The threshold concentration value of the target protein is determined experimentally and represents a value less than the aggregation concentration but higher than the standard biological concentration. Ostensibly one may perform the method in the absence of determining the concentration of the predetermined protein, say in the case of a family where a certain condition is common amongst the family members. Still, in most cases, the concentration of the predetermined protein is measured, and if higher than a predetermined threshold, then the patient in question will be encouraged to allow for selective filtering of the protein in question. The ports are placed where needed, depending which fluid is to be selectively filtered: blood, cerebrospinal fluid, spinal fluid, or other. The ports are adapted to allow for opening and closing as well as for attachment to sterile tubing. The tubing leads to and from a column with a stationary phase that is adapted to selectively bind the predetermined protein and effectively reduce the concentration of the predetermined protein prior to returning fluid to the second port and back into the body.

It is understood that the column, tubing and any ancillary components are for single sterile use and not for multiple application. These elements may be sterilized by any relevant means. One may measure the concentration of the predetermined protein in the relevant fluid immediately after passage of the fluid through the column or at a later time, depending on the diagnostic requirements for measuring the concentration of the predetermined protein.

### Fourth Embodiment

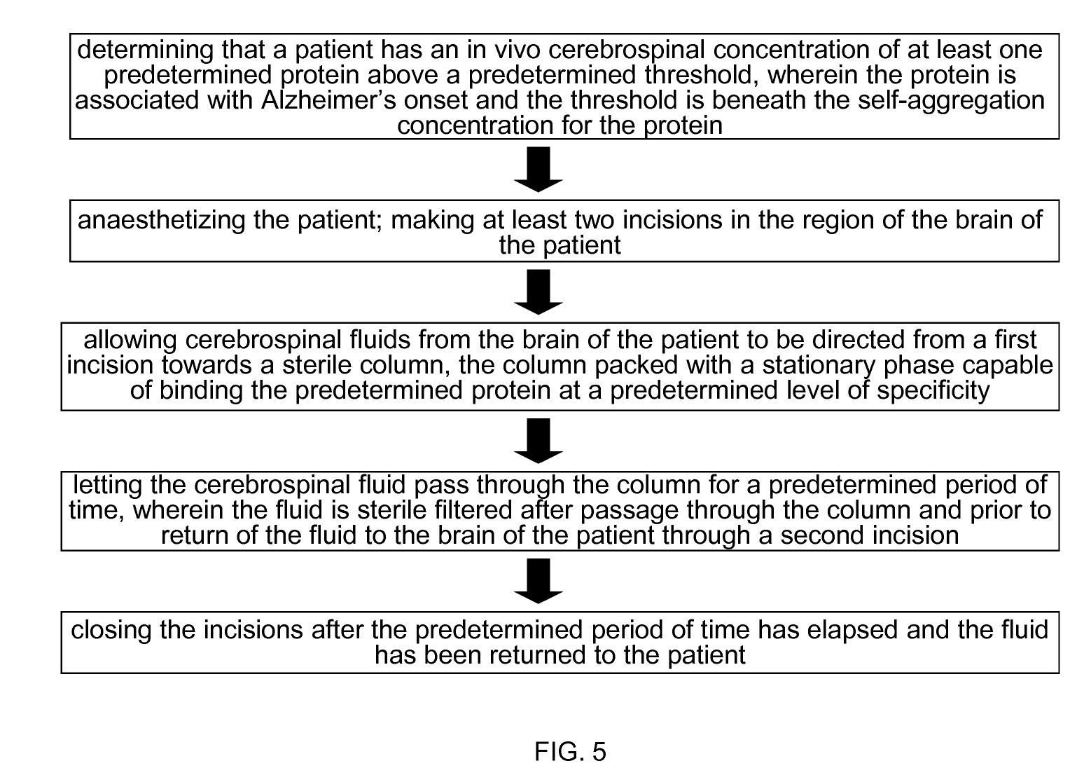

Attention is now turned to FIG. 5, which shows an embodiment of a method of the instant invention. The method includes the following: determining that a patient has an in vivo cerebrospinal concentration of at least one predetermined protein, such as beta-amyloid or tau, above a predetermined threshold, wherein the protein is associated with Alzheimer's onset and the threshold is beneath the self-aggregation concentration for the protein; anaesthetizing the patient; making at least two incisions in the region of the brain of the patient; allowing cerebrospinal fluids from the brain of the patient to be directed from a first incision towards a sterile column, the column packed with a stationary phase capable of binding the predetermined protein at a predetermined level of specificity; letting the cerebrospinal fluid pass through the column for a predetermined period of time, wherein the fluid is sterile filtered after passage through the column and prior to return of the fluid to the brain of the patient through a second incision; and, closing the incisions after the predetermined period of time has elapsed and the fluid has been returned to the patient. The instant embodiment is related to the prevention or treatment of Alzheimer's disease. There are tests today that “predict” the likelihood of a person getting Alzheimer's in the future. If the concentration of beta-amyloid, tau or other relevant protein is higher than desired and thus risks self-aggregation of protein on the brain, one could perform the instant method to prevent a patient from ever getting Alzheimer's. By “thinning out” the concentration of the problematic protein, the instant invention would prevent the undesirable self-aggregation of protein material to form amyloids, fibrils or other undesired protein superstructures. The incisions allow for the reversible introduction of tubing for the purpose of moving cerebrospinal fluid through the column for selective protein binding and removal, prior to return of cerebrospinal fluid to the patient.

### Fifth Embodiment

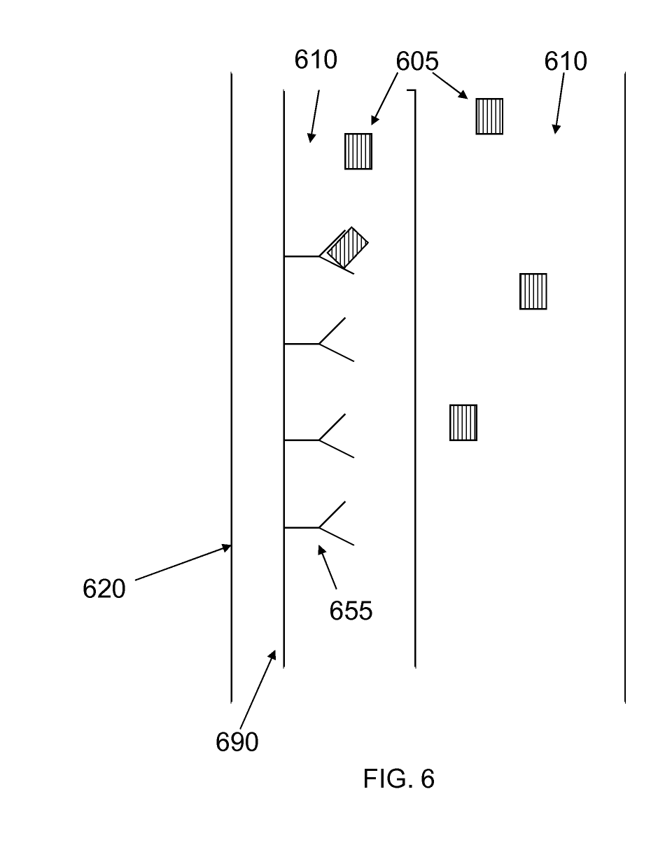

Attention is turned to FIG. 6. The present invention may be practiced as an in vivo implementation and device for accomplishing specific protein retention and removal. For example, a porous chamber 690 containing a stationary phase 625 with attached antibodies 655 specific to a disease-associated proteins 605 could be embedded in the body 620 in a location exposed to a biological fluid 610 containing said proteins 605. Passage of said biological fluid 610 through the chamber 690 would only reduce the concentration level of the predetermined undesired proteins 605. The removal capacity of the chamber 690 could be calibrated to match the desired level of protein 605 removal through prior determination of the initial protein 605 concentration, the volume of the biological fluid 610 in the body, and the desired level of final protein 605 concentration. An in vivo device of the present embodiment may involve internal pumps and other components (not shown) known to those who are skilled in the art, and may additionally include in vivo concentration measurement sensors and control devices. The chamber 690 could be represented by a column or dialysis membrane decorated with antibodies 655 or other appropriate element that allows flow of fluid 610 through the chamber 690.

### Example

A forty-five year old patient is checked for beta-amyloid concentration in his blood serum. The concentration is determined to be higher than usual, and his case history shows that both parents are Alzheimer's patients with advanced dementia. While the patient is showing no signs of dementia, it is determined to perform a beta amyloid reduction program according to an embodiment of the instant invention.

The patient is admitted to hospital and prepped for surgery on the back of his head. Two small incisions are made at the base of the skull and appropriate sterile stopcocks are inserted in the incision sites. A sterile chromatographic column with tubing is attacked to the stopcocks, wherein the stationary phase of the column is Sepharose® with 10% of the resin beads pre-modified to include goat antibodies with high affinity of binding for beta-amyloid monomers, while the mobile phase is phosphate buffer, pH 7.5. A small pump is attached to the tubing to force cerebrospinal fluid (CSF) from patient to flow in a single direction from the predetermined top of the column to its bottom. A filter is placed in the tubing after the column so as to guarantee that CSF returning to patient is at all times sterile. CSF is circulated for 6 hours through the column, 10 centimeters long, 1 centimeter in diameter, with the stationary and mobile phases encased in plastic. The patient is under general anesthetic during this time. After six hours, patient's beta amyloid concentration is checked and found to have been reduced by 90%. The tubing and column are removed and disposed of as biological waste, while the incisions are stitched closed and the patient is moved to a recovery room. After an overnight hospital stay, the patient is sent home. Every five years, said patient is checked for circulating beta amyloid concentration, and if the concentration should again be higher than desired, the procedure herewith described would be performed again.

The instant invention may be applied for any relevant biological fluid taken from a patient. A column according to the instant invention may bind a single or multiple unique protein targets. The protein targets may be selectively removed from the column for further research, but not for return to the patient. The column may be realized as a plurality of columns in parallel or series and the dimensions and shape of a column are selected so as to allow for efficient cleaning of a bodily fluid sample of the protein materials of interest. The bodily fluid before, during, or after passage through the column may be further treated if desired.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. The present invention could be employed for a wide variety of embodiments for human or veterinary purposes.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements. Certain embodiments of the instant invention may include design features that allow for faster and/or safer patient treatment. Methods of the instant invention may be performed under general or local anesthetic, if required.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed:

1. A method for treating a disease in a patient, including the following:
determining that a patient has an in vivo concentration of at least one predetermined protein above a predetermined threshold concentration, wherein said protein may be associated with disease;
creating at least two separated ports on said patients, allowing access to at least one biological fluid;
allowing a predetermined biological fluid from said patient to be directed from a first of said ports towards a sterile column, said column packed with a stationary phase capable of binding said predetermined protein at a predetermined level of specificity;
letting said fluid pass through said column for a predetermined period of time, thereby causing the concentration of the at least one predetermined protein in said fluid to be lowered below a predetermined threshold;
allowing said fluid to return to said patient through a second of said ports after fluid passage through said column; and,
closing said ports after said predetermined period of time has elapsed and said fluid has been returned to said patient.

2. The method according to claim 1, wherein said disease is selected from the following: Alzheimer's disease, Parkinson disease, Huntington disease, and Amyotropic Lateral Sclerosis disease.

3. The method according to claim 1, wherein said disease is a neurodegenerative disease.

4. The method according to claim 1, wherein said disease is selected from the list: Alzheimer's disease, aortic medial amyloid, atherosclerosis, cardiac arrhythmias, isolated atrial amyloidosis, cerebral amyloid angiopathy, diabetes mellitus type 2, dialsysis-related amyloidosis, familial amyloid polyneuropathy, Finnish amyloidosis, hereditary non-neuropathic systemtic amyloidosis, Huntington's disease, lattice corneal dystrophy, medullary carcinoma of the thyroid, multiple myeoloma, Parkinson's disease, prolactinomas, rheumatoid arthritis, sporadic inclusion body myositis, systemic AL amyloidosis, transmissible spongiform encephalopathy.

5. The method according to claim 2, wherein said predetermined protein is selected from the following: beta-amyloid, tau, and alpha-synuclein.

6. The method according to claim 1, wherein said stationary phase includes immobilized antibodies specific for interaction and binding of said predetermined protein.

7. The method according to claim 1, wherein said step of determining is performed in advance of said step of creating said access ports.

8. The method according to claim 1, wherein said threshold is below a concentration value required for protein aggregation.

9. The method according to claim 1, wherein said predetermined period of time is 3, 6, 12, or 24 hours.

10. The method according to claim 1, further including a step of measuring the concentration of said predetermined protein before said step of closing said ports.

11. The method according to claim 1, wherein said predetermined protein is realized as a plurality of unique proteins.

12. The method according to claim 1, wherein a pump is employed in said step of letting said biological fluid pass through said column and a filter is placed after said column.

13. A method for prophylactic treatment for Alzheimer's disease, including the following:
determining that a patient has an in vivo cerebrospinal concentration of at least one predetermined protein above a predetermined threshold, wherein said protein is associated with Alzheimer's onset and said threshold is beneath the self-aggregation concentration for said protein;
anaesthetizing said patient;
making at least two incisions in the region of the brain of said patient;
allowing cerebrospinal fluids from the brain of said patient to be directed from a first incision towards a sterile column, said column packed with a stationary phase capable of binding said predetermined protein at a predetermined level of specificity;
letting said cerebrospinal fluid pass through said column for a predetermined period of time, wherein said fluid is filtered after passage through said column and prior to return of said fluid to the brain of said patient through a second incision; and,
closing said incisions after said predetermined period of time has elapsed and said fluid has been returned to said patient.

14. The method according to claim 13, wherein said determining is performed on a patient age of 40 or higher.

15. The method according to claim 13, wherein said method is performed on a patient more than once in his life.

16. The method according to claim 15, wherein said method is performed every five or ten years.

17. A method for treating a disease in a patient, including the following:
determining that a patient has an in vivo concentration of at least one predetermined protein above a predetermined threshold concentration, wherein said protein may be associated with disease;
implanting a chamber in the body of said patient, wherein said chamber allows for flow of a bodily fluid through said chamber;
allowing a predetermined biological fluid from said patient to be directed to flow through said chamber, said chamber including a stationary phase capable of binding said predetermined protein at a predetermined level of specificity;
letting said fluid pass through said chamber for a predetermined period of time, thereby causing the concentration of the at least one predetermined protein in said fluid to be lowered below a predetermined threshold; and,
monitoring the in vivo concentration of said predetermined protein.

18. The method according to claim 17, further including at least one in vivo pump to direct the flow of said bodily fluid through said chamber.

19. The method according to claim 17, further including at least one in vivo sensor to monitor the concentration of said at least one predetermined protein.

20. The method according to claim 17, further including the step of removing said chamber.
